(19) United States
(12) Patent Application Publication
(10) Pub. No.: US 2026/0354861 A1
(43) Pub. Date: Nov. 12, 2026

(54) ANCHOR FOR TRANSCATHETER ACCESS INTO THE CORONARY SINUS

(71) Applicant: Edwards Lifesciences Corporation, Irvine, CA (US)

(72) Inventors: Linda Thai, Mission Viejo, CA (US); Michael G. Valdez, Riverside, CA (US); Tarannum Ishaq Gutierrez, Ladera Ranch, CA (US); Don Huy Tran, Westminster, CA (US)

(21) Appl. No.: 19/642,425

(22) Filed: Apr. 8, 2026

Related U.S. Application Data
(62) Division of application No. 17/507,734, filed on Oct. 21, 2021, now Pat. No. 12,678,191.
Application No. 17/507,734 is a continuation of application No. PCT/US2020/027147, filed on Apr. 8, 2020.
(60) Provisional application No. 62/836,851, filed on Apr. 22, 2019.

(51) Int. Cl. A61B 17/34; A61B 17/00; A61M 25/10

(52) U.S. Cl. CPC A61B 17/3423; A61B 17/3468; A61B 2017/00243; A61B 2017/00292; A61B 2017/3425; A61B 2017/3486; A61M 25/1002

(57) ABSTRACT

A delivery sheath can comprise an elongate tubular member and an anchor on a distal portion of the elongate tubular member, the anchor being configured to engage with the coronary sinus ostium, or both the coronary sinus ostium and a portion of the coronary sinus adjacent to the ostium, so as to provide stable transcatheter access into the coronary sinus. The anchor can be configured to permit blood flow through the ostium while in engagement with the ostium.

### CROSS REFERENCE TO RELATED APPLICATIONS

This application is a divisional application of U.S. application Ser. No. 17/507,734, filed Oct. 21, 2021 and entitled ANCHOR FOR TRANSCATHETER ACCESS INTO THE CORONARY SINUS, which is a continuation application of International Patent Application Serial No. PCT/US2020/027147, filed Apr. 8, 2020 and entitled ANCHOR FOR TRANSCATHETER ACCESS INTO THE CORONARY SINUS, which claims priority based on U.S. Provisional Patent Application Ser. No. 62/836,851, filed Apr. 22, 2019 and entitled ANCHOR FOR TRANSCATHETER ACCESS INTO THE CORONARY SINUS, the full disclosures of both of which are hereby expressly incorporated by reference herein in their entireties for all purposes.

### BACKGROUND

### Field

The present disclosure generally relates to the field of delivering medical implant devices and/or therapies.

### Description of Related Art

Transcatheter delivery of implant devices and/or therapies to the heart can be performed to address various heart abnormalities. Delivery of implant devices and/or therapies to the heart can be performed for treatment of elevate pressure in the left atrium.

### SUMMARY

Described herein are methods and devices for providing stable transcatheter access into the coronary sinus from the right atrium via the coronary sinus ostium. In some implementations, the present disclosure relates to a steerable sheath comprising an elongate tubular member which includes an inner lumen configured to receive a plurality of instruments, and a partially circumferential balloon anchor disposed partially circumferentially on a distal portion of the elongate tubular member. The partially circumferential balloon anchor can include a proximal balloon portion and a distal balloon portion. When the partially circumferential balloon anchor is in an inflated state, the distal balloon portion can have a tapered longitudinal profile tapering toward a distal end of the partially circumferential balloon anchor, and the partially circumferential balloon anchor can be configured to, while positioned to engage with a coronary sinus, permit blood flow through an ostium of the coronary sinus.

In some embodiments, the partially circumferential balloon anchor, in the inflated state, is configured to be positioned to engage with both the ostium and a portion of the coronary sinus adjacent to the ostium.

In some embodiments, the tapered longitudinal profile comprises a convex curvature.

In some embodiments, the partially circumferential balloon anchor is positioned around less than 50% of a circumference of the distal portion of the elongate tubular member.

In some embodiments, the partially circumferential balloon anchor comprises a textured exterior surface configured to engage with the ostium and a portion of the coronary sinus adjacent to the ostium. The textured exterior surface can comprise either or both of a plurality of bumps and a plurality of ridges.

In some embodiments, the proximal balloon portion extends laterally from the distal portion of the elongate tubular member, a deformable panel being coupled to the proximal balloon portion.

In some embodiments, the inner lumen is configured to receive a therapy device delivery catheter carrying a therapy device for deployment to a heart location. The therapy device can comprise a shunt device for deployment onto a left atrial wall.

In some implementations, a steerable sheath comprises an elongate tubular member which includes an inner lumen configured to receive a plurality of instruments, and a circumferential balloon anchor disposed circumferentially around a distal portion of the elongate tubular member. A plurality of cords can be circumferentially distributed around the circumferential balloon anchor. When the circumferential balloon anchor is in an inflated state, the plurality of cords can be configured to engage the circumferential balloon anchor to create corresponding circumferentially distributed indentations on the circumferential balloon anchor, and the circumferential balloon anchor can be configured to, while positioned to engage with a coronary sinus, permit blood flow through an ostium of the coronary sinus along a path defined at least in part by the circumferentially distributed indentations.

In some embodiments, the circumferential balloon anchor, in the inflated state, is configured to be positioned to engage with the ostium.

In some embodiments, the circumferential balloon anchor comprises a proximal balloon portion and a distal balloon portion, and respective distal ends of the plurality of cords are coupled to corresponding locations on the distal balloon portion, the plurality of cords being movable relative to the elongate tubular member along a longitudinal axis of the elongate tubular member. Moving the plurality of cords proximally relative to the elongate tubular member can be configured to engage the circumferential balloon anchor in the inflated state with the plurality of cords to provide the corresponding circumferentially distributed indentations. In some embodiments, the plurality of cords comprises five cords.

In some embodiments, the distal balloon portion comprises a tapered longitudinal profile tapering toward a distal end of the circumferential balloon anchor, the distal balloon portion being configured to engage with the ostium and a portion of the coronary sinus adjacent to the ostium.

In some embodiments, the circumferential balloon anchor comprises a textured exterior surface configured to engage with the ostium and a portion of the coronary sinus adjacent to the ostium. The textured exterior surface can comprise either or both of a plurality of bumps and a plurality of ridges.

In some embodiments, the inner lumen is configured to receive a therapy device delivery catheter carrying a therapy device for deployment to a heart location. The therapy device can comprise a shunt device for deployment onto a left atrial wall.

In some implementations, a method of accessing a heart location can comprise providing a steerable sheath which includes an elongate tubular member, a circumferential balloon anchor circumferentially positioned around a distal portion of the elongate tubular member and a plurality of cords circumferentially distributed around the circumferential balloon anchor. The distal portion of the elongate tubular member can be introduced through an inferior vena cava or a superior vena cava, and into a right atrium. A distal end of the elongate tubular member can be inserted through an ostium of a coronary sinus into the coronary sinus from the right atrium, and the distal end can be positioned at a target location within the coronary sinus. The circumferential balloon anchor can be inflated and an inflated circumferential balloon anchor can be engaged with the plurality of cords to create corresponding circumferentially distributed indentations on the inflated circumferential balloon anchor. The inflated circumferential balloon anchor can be engaged with the coronary sinus while blood flow is permitted through the ostium past the inflated circumferential balloon anchor along a path defined at least in part by the circumferentially distributed indentations.

In some embodiments, engaging the coronary sinus comprises engaging the inflated circumferential balloon anchor with the ostium of the coronary sinus.

In some embodiments, the plurality of cords can be moved proximally along a longitudinal axis of the elongate tubular member, respective distal ends of the plurality of cords being coupled to corresponding locations on a distal balloon portion, where moving the plurality of cords compresses the inflated circumferential balloon anchor to provide the corresponding circumferentially distributed indentations on the inflated circumferential balloon anchor.

In some embodiments, a delivery catheter carrying a therapy device can be inserted into an inner lumen of the elongate tubular member. A distal end of the delivery catheter can be advanced from the distal end of the elongate tubular member for positioning at the target location. The delivery catheter can comprise a shunt delivery catheter carrying an atrial shunt device.

In some implementations, a steerable sheath can comprise an elongate tubular member which includes an inner lumen configured to receive a plurality of instruments, and a deformable helical rib anchor disposed around a distal portion of the elongate tubular member, the deformable helical rib anchor being configured to engage with a coronary sinus, and to permit blood flow through an ostium of the coronary sinus along a path defined at least in part by the deformable helical rib anchor while the deformable helical rib is positioned to engage with the coronary sinus.

In some embodiments, the deformable helical rib anchor is configured to be positioned to engage with both the ostium of the coronary sinus and a portion of the coronary sinus adjacent to the ostium.

In some embodiments, a height at which the deformable helical rib anchor extends from the distal portion of the elongate tubular member increases with increased distance from a distal end of the elongate tubular member. In some embodiments, a height at which the deformable helical rib anchor extends from the distal portion of the elongate tubular member is uniform along a dimension of the deformable helical rib anchor parallel with a longitudinal axis of the elongate tubular member.

In some embodiments, the inner lumen is configured to receive a therapy device delivery catheter carrying a therapy device for deployment to a heart location. The therapy device can comprise a shunt device for deployment onto a left atrial wall.

In some implementations, a steerable sheath can comprise an elongate tubular member which includes an inner lumen configured to receive a plurality of instruments, and a first expandable coil anchor disposed around a distal portion of the elongate tubular member, the first expandable coil being configured to engage with a coronary sinus and to permit blood flow through an ostium of the coronary sinus while the first expandable coil anchor is in an expanded state and positioned to engage with the coronary sinus.

In some embodiments, the first expandable coil is configured to be positioned to engage with the ostium of the coronary sinus.

In some embodiments, the steerable sheath further comprises a second expandable coil anchor positioned distally adjacent to the first expandable coil anchor, the second expandable coil anchor comprising a diameter smaller than that of the first expandable coil anchor, the second expandable coil anchor being configured to engage with a portion of the coronary ostium adjacent to the ostium when in an expanded state. The first expandable coil anchor and the second expandable coil anchor can be configured to permit blood flow through the ostium while the first expandable coil anchor and the second expandable coil anchor are positioned to engage with the ostium and the portion of the coronary sinus adjacent to the ostium.

In some embodiments, the inner lumen can be configured to receive a therapy device delivery catheter carrying a therapy device for deployment to a heart location. The therapy device can comprise a shunt device for deployment onto a left atrial wall.

In some implementations, a steerable sheath can comprise an elongate tubular member which includes an inner lumen configured to receive a plurality of instruments, and a partially circumferential ring anchor disposed partially circumferentially on a distal portion of the elongate tubular member, the partially circumferential ring anchor being configured to permit blood flow through an ostium of a coronary sinus while the partially circumferential ring anchor is positioned to engage with the coronary sinus.

In some embodiments, the partially circumferential ring anchor is configured to be positioned to engage with the ostium.

In some embodiments, the partially circumferential ring anchor comprises a proximal ring portion and a distal ring portion, the distal ring portion comprising a tapered longitudinal profile tapering toward a distal end of the ring, the partially circumferential ring anchor being configured to engage with the ostium and a portion of the coronary sinus adjacent to the ostium.

In some embodiments, the partially circumferential ring is disposed around less than 50% of a circumference of the distal portion of the elongate tubular member.

In some embodiments, the inner lumen is configured to receive a therapy device delivery catheter carrying a therapy device for deployment to a heart location. The therapy device can comprise a shunt device for deployment onto a left atrial wall.

In some implementations, a method of accessing a heart location can comprise providing a steerable sheath which includes an elongate tubular member and an anchor positioned on a distal portion of the elongate tubular member. The distal portion of the elongate tubular member can be introduced through an inferior vena cava or a superior vena cava, and into a right atrium. A distal end of the elongate tubular member can be inserted through an ostium of a coronary sinus into the coronary sinus from the right atrium, and the distal end can be positioned at a target location within the coronary sinus. The anchor can be engaged with the coronary sinus while blood flow is permitted through the ostium past the anchor.

In some embodiments, engaging the coronary sinus comprises engaging the ostium of the coronary sinus with the anchor.

In some embodiments, a delivery catheter carrying a therapy device can be inserted into an inner lumen of the elongate tubular member, and a distal end of the delivery catheter can be advanced from the distal end of the elongate tubular member for positioning at the target location. The delivery catheter carrying a therapy device can comprise a shunt delivery catheter carrying an atrial shunt device

For purposes of summarizing the disclosure, certain aspects, advantages and novel features have been described herein. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements. However, it should be understood that the use of similar reference numbers in connection with multiple drawings does not necessarily imply similarity between respective embodiments associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some embodiments or configurations.

FIG. 1 is a cross-sectional view of a human heart.

FIG. 2 is another cross-sectional view of the human heart.

FIGS. 3A and 3B show examples of pathways for accessing the coronary sinus from the right atrium.

FIG. 4 is a schematic diagram of an example of a delivery system comprising a steerable sheath and a delivery catheter received within an inner lumen of the steerable sheath.

FIG. 5 is a schematic diagram of the steerable sheath of FIG. 4 positioned from a first body chamber or lumen into a second body chamber or lumen.

FIG. 6 is a schematic diagram of a side view of an example of a steerable sheath comprising a partially circumferential balloon anchor.

FIG. 7 is a schematic diagram of a view of the distal portion of the steerable sheath of FIG. 6.

FIG. 8 shows the steerable sheath of FIG. 6 positioned for accessing a target location within the coronary sinus.

FIG. 9 is a schematic diagram of an example of a steerable sheath comprising a partially circumferential balloon anchor and a deformable panel coupled thereto.

FIGS. 10A and 10B are schematic diagrams of examples of steerable sheaths which comprise a partially circumferential balloon anchor, where the partially circumferential balloon anchor can comprise a textured surface thereon.

FIGS. 11A and 11B are schematic diagrams showing an example of a steerable sheath which comprises a circumferential balloon anchor and a plurality of cords circumferentially distributed around the circumferential balloon anchor.

FIGS. 12A and 12B are schematic diagrams showing the plurality of cords engaged with the circumferential balloon anchor of FIGS. 11A and 11B, so as to form corresponding indentations on the circumferential balloon anchor.

FIGS. 13A and 13B are schematic diagrams showing examples of steerable sheaths comprising a helical rib anchor.

FIGS. 14A and 14B are schematic diagrams showing examples of steerable sheaths comprising one coil anchor and two coil anchors, respectively.

FIG. 15 is a schematic diagram showing an example of a steerable sheath comprising a partially circumferential ring anchor.

FIG. 16 is a flow diagram of an example of a procedure for deploying a steerable sheath into the coronary sinus.

FIG. 17 is a flow diagram of an example of a procedure for deploying a steerable sheath comprising a circumferential balloon anchor into the coronary sinus.

### DETAILED DESCRIPTION

The headings provided herein are for convenience only and do not necessarily affect the scope or meaning of the claimed invention.

The present disclosure relates to systems, devices, and methods for providing reliable positioning of delivery sheaths into the coronary sinus from the right atrium via the coronary sinus ostium. The steerable sheaths can comprise an anchor configured to engage with the coronary sinus ostium, or both the coronary sinus ostium and a portion of the coronary sinus adjacent to the ostium, for providing stable transcatheter access into the coronary sinus.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred embodiments. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

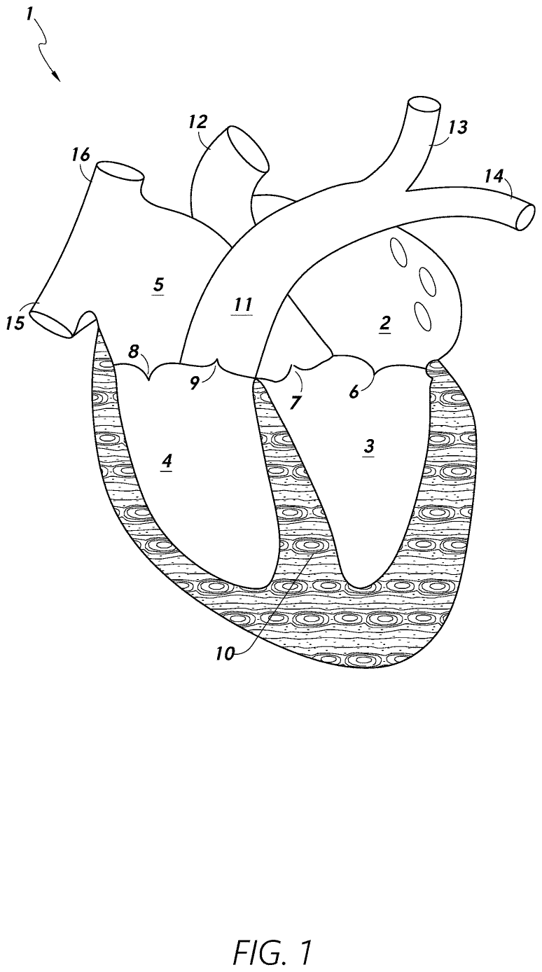

Various features of a heart 1 are described with reference to FIG. 1 to assist in understanding the present disclosure. The heart 1 includes four chambers, namely the left atrium 2, the left ventricle 3, the right ventricle 4, and the right atrium 5. A wall of muscle, referred to as the septum 10, separates the left atrium 2 and right atrium 5, and the left ventricle 3 and right ventricle 4. Blood flow through the heart 1 is at least partially controlled by four valves, the mitral valve 6, aortic valve 7, tricuspid valve 8, and pulmonary valve 9. The mitral valve 6 separates the left atrium 2 and the left ventricle 3 and controls blood flow therebetween. The aortic valve 7 separates and controls blood flow between the left ventricle 3 and the aorta 12. The tricuspid valve 8 separates the right atrium 5 and the right ventricle 4 and controls blood flow therebetween. The pulmonary valve 9 separates the right ventricle 4 and the pulmonary artery 11, controlling blood flow therebetween.

In a healthy heart, the heart valves can properly open and close in response to a pressure gradient present during various stages of the cardiac cycle (e.g., relaxation and contraction) to at least partially control the flow of blood to a respective region of the heart and/or to blood vessels. Deoxygenated blood arriving from the rest of the body generally flows into the right side of the heart for transport to the lungs, and oxygenated blood from the lungs generally flows into the left side of the heart for transport to the rest of the body. During ventricular diastole, deoxygenated blood arrive in the right atrium 5 from the inferior vena cava 15 and superior vena cava 16 to flow into the right ventricle 4, and oxygenated blood arrive in the left atrium 2 from the pulmonary veins to flow into the left ventricle 3. During ventricular systole, deoxygenated blood from the right ventricle 4 can flow into the pulmonary artery 11 for transport to the lungs (e.g., via the left 14 and right 13 pulmonary arteries), and oxygenated blood can flow from the left ventricle 3 to the aorta 12 for transport to the rest of the body.

A number of conditions can contribute to a higher than normal pressure in the left atrium. Dysfunction of the mitral valve can contribute to elevated left atrial pressure. Conditions such as mitral valve regurgitation and/or stenosis may result in difficulty in pumping blood from the left atrium to the left ventricle, contributing to elevated pressure in the left atrium. Valve stenosis can cause a valve to become narrowed or obstructed. Mitral valve stenosis can restrict blood flow from the left atrium to the left ventricle. Valve regurgitation occurs when a valve does not close properly. For example, regurgitation can occur due to improper coaptation of the valve leaflets. Mitral valve regurgitation can result in blood flow leakage back into the left atrium 2 from the left ventricle 3 when the left ventricle 3 contracts. Restricted flow of blood from the left atrium 2 into the left ventricle 3, and blood flow leakage from the left ventricle 3 back into the left atrium 2 can both contribute to elevated atrial pressure. Dysfunction in the left ventricle 3 can also contribute to elevated left atrial pressure. Elevated left atrial pressure may lead to left atrial enlargement, producing symptoms such as shortness of breath during exertion, fatigue, chest pain, fainting, abnormal heartbeat, and swelling of the legs and feet.

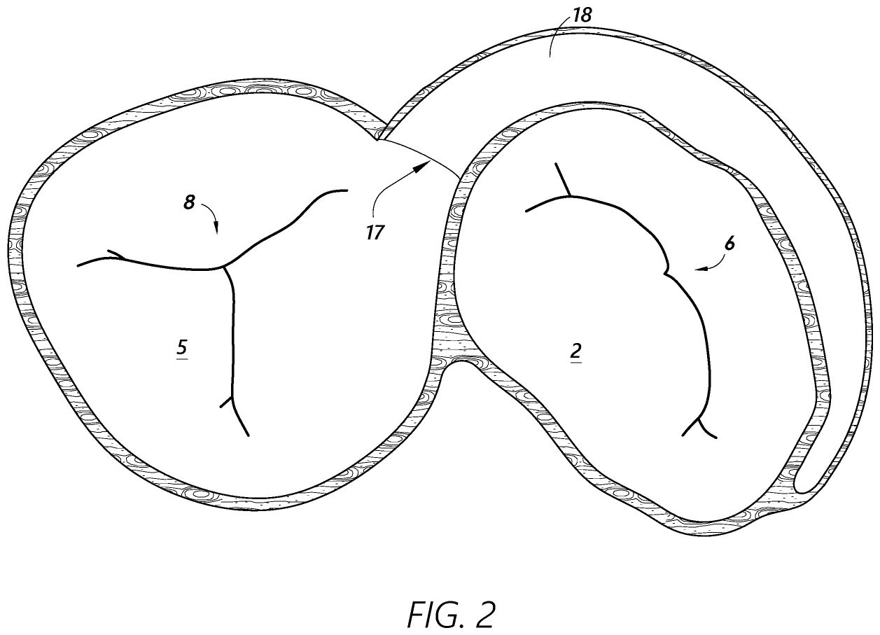

FIG. 2 is another view of the heart 1 and shows the coronary sinus 18 around the left atrium 2. To alleviate elevated left atrial pressure, a conduit can be provided to allow blood flow from the left atrium 2 into a portion of the heart with lower pressure, such as the coronary sinus 18. A conduit can be formed on the wall of the left atrium 2 adjacent to the coronary sinus 18 to allow blood flow from the left atrium 2 into the coronary sinus 18. The coronary sinus 18 receives blood from coronary veins and empties into the right atrium 5. Blood diverted into the coronary sinus 18 from the left atrium 2 can then be delivered into the right atrium 5. A shunt device can be positioned at a location on the left atrial wall, such as at a location on the left atrial wall which is accessible from the coronary sinus 18, to form a blood flow pathway from the left atrium 2 into the coronary sinus 18. Access into the coronary sinus 18 can comprise navigating into the right atrium 5 and entering through the coronary sinus ostium 17.

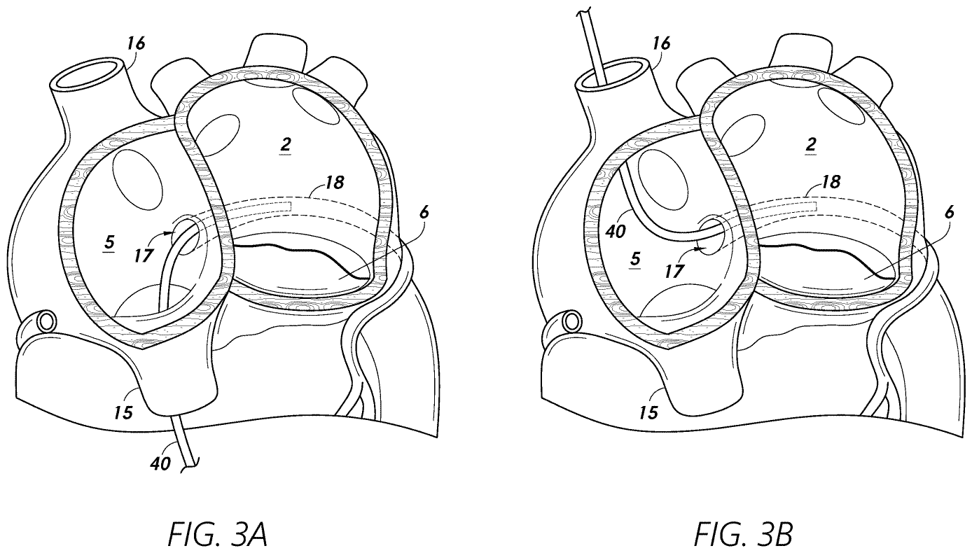

FIGS. 3A and 3B show examples of pathways for accessing the coronary sinus 18 through the right atrium 5 to deliver a shunt or other implant device to a portion of the left atrial wall. In FIG. 3A, a steerable sheath 40 is shown as being inserted through the inferior vena cava 15 and into the right atrium 5. For example, the steerable sheath 40 can be introduced through the femoral vein, and then inserted through the inferior vena cava 15. The steerable sheath 40 can then be advanced from the right atrium 5 through the coronary sinus ostium 17 and into the coronary sinus 18.

In FIG. 3B, the steerable sheath 40 is shown as being inserted through the superior vena cava 16 and into the right atrium 5. The steerable sheath 40 can be introduced through the subclavian vein or jugular vein, and then inserted through the superior vena cava 16. The steerable sheath 40 can be positioned from the right atrium 5 through the coronary sinus ostium 17 and into the coronary sinus 18.

As shown in both FIGS. 3A and 3B, the steerable sheath 40 can be bent when positioned from the inferior vena cava 15 or the superior vena cava 16 into the right atrium 5 and from the right atrium 5 through the coronary sinus ostium 17. This curvature in the steerable sheath 40 can render stable positioning of the steerable sheath 40 difficult when moving instrumentation through an inner lumen of the steerable sheath 40 to position instrumentation within the coronary sinus 18. Advancing and/or retracting instrumentation through the steerable sheath 40, and/or rigidity of the steerable sheath 40, may inadvertently move the steerable sheath 40 and dislodge the steerable sheath 40 from its desired position. Movement of instrumentation within the steerable sheath 40 can push a distal end of the steerable sheath 40 positioned within the coronary sinus 18 back toward the coronary sinus ostium 17.

In some embodiments, the present disclosure relates to devices, systems and/or methods for providing stable transcatheter access into the coronary sinus from the right atrium via the coronary sinus ostium. Described herein are delivery sheaths, including steerable sheaths, for positioning into the coronary sinus, the steerable sheaths comprising an anchor configured to engage with the coronary sinus ostium, or both the coronary sinus ostium and a portion of the coronary sinus adjacent to the ostium. The anchor can be on a distal portion of an elongate tubular member of the steerable sheath, a distal end of the steerable sheath being configured to be positioned within the coronary sinus for accessing the left atrial wall. The anchor can allow stable positioning of the distal end within the coronary sinus to facilitate reliable delivery of one or more medical implant devices and/or therapies to a target tissue site on the left atrial wall.

As described in further detail herein, an elongate tubular member of a steerable sheath can be bent when positioned through the coronary sinus ostium from the right atrium. The steerable sheath can be inserted into the right atrium through the inferior vena cava or superior vena cava. The bend or curvature in the elongate tubular member can present difficulties in maintaining a stable and secure position within the coronary sinus. Insertion and/or retraction of instrumentation through the elongate tubular member, such as to deliver implant devices and/or therapies to the left atrial wall, can cause undesired movement of the distal portion of the elongate tubular member. The distal portion may be pushed back towards the right atrium, dislodging the elongate tubular member from its desired location within the coronary sinus.

Engaging an anchor with the coronary sinus ostium, or with both the coronary sinus ostium and an adjacent portion of the coronary sinus, can facilitate docking of the elongate tubular member against the coronary sinus ostium, or the coronary sinus ostium and an adjacent portion. Force can be exerted upon the elongate tubular member to maintain the distal portion of the elongate tubular member at its desired position within the coronary sinus. Engaging the anchor with the coronary sinus ostium, or the coronary sinus ostium and an adjacent portion, can prevent over insertion of the elongate tubular member into the coronary sinus, while reducing or avoiding movement of the elongate tubular member back towards the right atrium. The anchor can provide secure positioning of the elongate tubular member within the coronary sinus such that desired therapy device implants and/or therapeutic procedures can be delivered thereto. Engaging the anchor with the coronary sinus ostium can provide improved stability and/or reduce or prevent damage to the coronary sinus, such as compared to steerable sheaths comprising anchors for engaging only with portions within the coronary sinus.

An anchor can be positioned on a distal portion of an elongate tubular member, spaced from or at a distal end of the elongate tubular member. The anchor can comprise a shape configured to engage with the coronary sinus ostium, or the coronary sinus ostium and an adjacent portion. A cross-sectional size of the coronary sinus decreases along a direction extending into the coronary sinus and away from the ostium. In some embodiments, at least a portion of the anchor can comprise a taper configured to engage with the coronary sinus ostium and an adjacent portion of the coronary sinus. For example, a distal portion of the anchor can have a longitudinal profile comprising a taper toward a distal end of the anchor, the longitudinal profile extending along a direction parallel or substantially parallel to a longitudinal axis of the elongate tubular member. In some embodiments, the tapered distal portion can comprise a curvature, including a convex curvature. In some embodiments, an anchor can comprise a textured exterior surface configured to provide desired engagement with the coronary sinus ostium and/or an adjacent portion of the coronary sinus.

The anchor can be configured to be in engagement with the coronary sinus ostium and an adjacent portion of the coronary sinus, while permitting blood flow through the ostium past the anchor. Permitting blood flow through the ostium can advantageously allow performance of delivery procedures while a heart is beating, providing a lower risk of complications, shorter hospital stay and/or quicker recovery for the patient. For example, the anchor can be configured so as not to occlude the ostium. In some embodiments, the anchor can comprise any one of a partially circumferential balloon anchor, a circumferential balloon anchor comprising longitudinal indentations formed thereon, a helical rib anchor, a coil anchor, a partially circumferential ring anchor, and a suction anchor. As used herein, a partially circumferential anchor can refer to any anchor configured to be positioned on, disposed around and/or extending from, any partial portion of the circumference of a distal portion of an elongate tubular member, and where the anchor does not circumscribe the distal portion.

Although the description herein is made primarily with reference to positioning sheaths from the right atrium into the coronary sinus, it will be understood that the description can refer or generally apply to positioning of sheaths from a first body chamber or lumen into a second body chamber or lumen, where the sheaths may be bent when positioned from the first body chamber or lumen into the second body chamber or lumen. A body chamber or lumen can refer to any one of a number of fluid channels, blood vessels, and/or organ chambers (e.g., heart chambers). Additionally, reference herein to “sheaths”, “delivery sheaths” and/or “steerable sheaths” can refer or apply generally to any type of elongate tubular delivery device comprising an inner lumen configured to slidably receive instrumentation, such as for positioning within an atrium or coronary sinus. “Sheaths”, “delivery sheaths” and/or “steerable sheaths” can include, for example, delivery catheters and/or cannulas. It will be understood that other types of medical implant devices and/or procedures can be delivered to the coronary sinus using a steerable sheath as described herein, including for example ablation procedures, drug delivery and/or placement of coronary sinus leads.

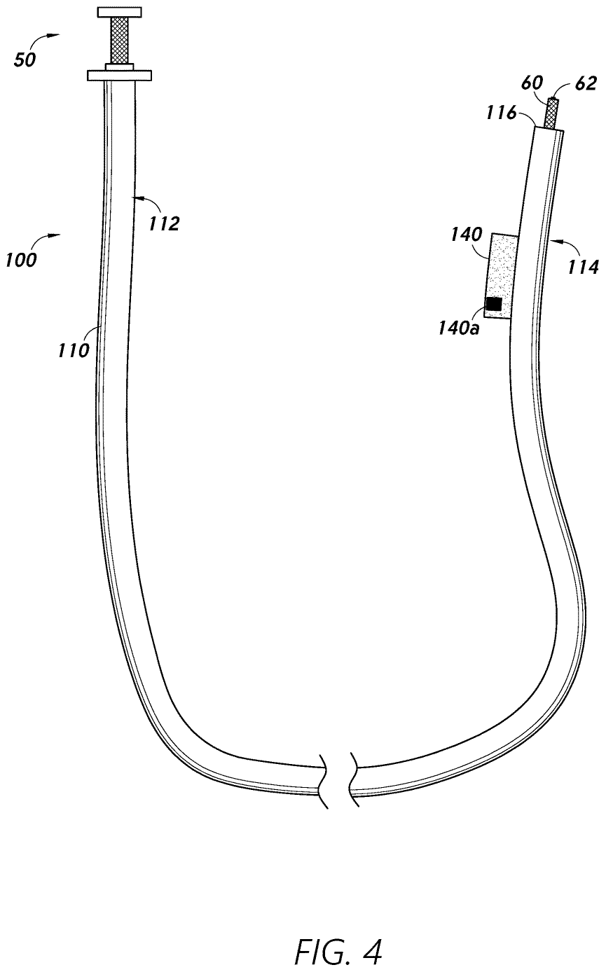

FIG. 4 is a schematic diagram of a delivery system 50 which comprises a steerable sheath 100 that includes an anchor 140. The steerable sheath 100 can comprise an elongate tubular member 110 having a proximal portion 112 and a distal portion 114. The anchor 140 can be on the distal portion 114 of the elongate tubular member 110. Various examples of the anchor 140 are described herein. For example, the anchor 140 can comprise any one of a partially circumferential balloon anchor, a circumferential balloon anchor having longitudinal indentations formed thereon, a helical rib anchor, a coil anchor, a partially circumferential ring anchor, and a suction anchor, as described herein.

The steerable sheath 100 can be configured to facilitate positioning of instrumentation at a target site within various body chambers or lumens. In some embodiments, the steerable sheath 100 can be configured to facilitate positioning of instrumentation at a target site within the coronary sinus, including a target tissue site on the left atrial wall accessible from within the coronary sinus. A delivery catheter 60 can be slidably received within an inner lumen of the elongate tubular member 110. A distal end 116 of the elongate tubular member 110 can be placed at or proximate to a target tissue site, including a target site in the coronary sinus. A distal end 62 of the delivery catheter 60 can be extended from the distal end 116 of the elongate tubular member 110 to position the distal end 62 of the delivery catheter 60 at or proximate to the target tissue site, such as to deploy one or more therapy devices to the target tissue site. In some embodiments, the delivery catheter 60 can be configured to carry one or more therapy devices for treatment of elevated left atrial pressure, such as an atrial shunt device. In some embodiments, the distal end 62 of the delivery catheter 60 can be positioned at or adjacent to a target site on the left atrial wall so as to facilitate deployment of an atrial shunt device onto the left atrial wall. It will be understood that other instrumentation can be received within the inner lumen, in addition to or instead of the delivery catheter 60.

In some embodiments, the steerable sheath 100 can be used to access the left atrial wall from within the coronary sinus by entering the coronary sinus from the right atrium through the coronary sinus ostium. As described herein, access into the right atrium can be made through the inferior vena cava or the superior vena cava. For example, the distal portion 114 of the elongate tubular member 110 can be configured to be advanced into the right atrium from the inferior vena cava or the superior vena cava. The distal portion 114 can then be inserted through the coronary sinus ostium from the right atrium, and into the coronary sinus. In some embodiments, the anchor 140 on the distal portion 114 can be configured to engage with the coronary sinus ostium. In some embodiments, the anchor 140 can be configured to engage with the coronary sinus ostium and a portion of the coronary sinus adjacent to the ostium. The anchor 140 can comprise a shape and/or size to provide desired engagement with the coronary sinus ostium, or the coronary sinus ostium and a portion of the coronary sinus adjacent to the ostium, while permitting flow of blood through the ostium past the anchor 140.

The anchor 140 can be at or a distance away from the distal end 116 of the elongate tubular member 110. In some embodiments, the anchor 140, such as a distal end of the anchor 140, can be at a distance of less than about 5 centimeters (cm) from the distal end 116 of the elongate tubular member 110, or less than about 4 cm, about 3 cm, about 2 cm, or about 1 cm. In some embodiments, the anchor 140 can be about 1 cm, about 2 cm, about 3 cm, or about 4 cm, from the distal end 116. In some embodiments, the anchor 140 can be at the distal end 116.

In some embodiments, the anchor 140 can comprise one or more radiopaque markers 140a to facilitate visualization of the positioning of the anchor 140, the distal portion 114 and/or the distal end 116 of the elongate tubular member 110. For example, the radiopaque markers 140a can facilitate desired positioning of the anchor 140 at the coronary sinus ostium. In some embodiments, the one or more radiopaque markers 140a can provide visualization of a shape, orientation, and/or location of the anchor 140 to facilitate correct positioning of the anchor 140 at the coronary sinus ostium, including visualization of a desired deformation of the anchor 140. For example, visualization of the deformation of a partially circumferential balloon anchor, a circumferential balloon anchor, and/or a helical rib anchor can be used to determine whether desired contact is made with the coronary sinus ostium and/or an adjacent portion of the coronary sinus.

As described herein, the anchor 140 can be configured to engage with the coronary sinus ostium, or the coronary sinus ostium and a portion of the coronary sinus adjacent to the ostium, to reduce or prevent undesired movement of the elongate tubular member 110. The anchor 140 can be configured to prevent dislodging of the elongate tubular member 110 from its desired position within the coronary sinus, such as when instrumentation is inserted and/or retracted through the inner lumen of the elongate tubular member 110. Reliable positioning of the elongate tubular member 110 within the coronary sinus can facilitate delivery of therapy device implants and/or application of therapeutic treatments to target sites accessible from within the coronary sinus, including target sites on the left atrial wall.

The anchor 140 can be configured to be in engagement with the coronary sinus ostium, or the coronary sinus ostium and an adjacent portion of the coronary sinus, while permitting blood flow through the ostium past the anchor 140. The anchor 140 can be configured so as not to occlude the ostium. Permitting blood flow through the ostium can have various advantages, including performance of procedures while a heart is beating. Beating heart surgeries can provide a lower risk of complications, shorter hospital stay and/or quicker recovery for the patient.

In some embodiments, the anchor 140 can be an integral part of the elongate tubular member 110. In some embodiments, the anchor 140 can be separable from the elongate tubular member 110. For example, the anchor 140 can be a separate component associated with the elongate tubular member 110 where the anchor 140 is configured to be coupled to the distal portion 114 of the elongate tubular member 110. The anchor 140 can be selected for coupling to the elongate tubular member 110. For example, a desired anchor can be selected for positioning onto the elongate tubular member 110 based on a number of factors, including a size and/or shape of the coronary sinus ostium and/or a distance from the ostium of the target site within the coronary sinus.

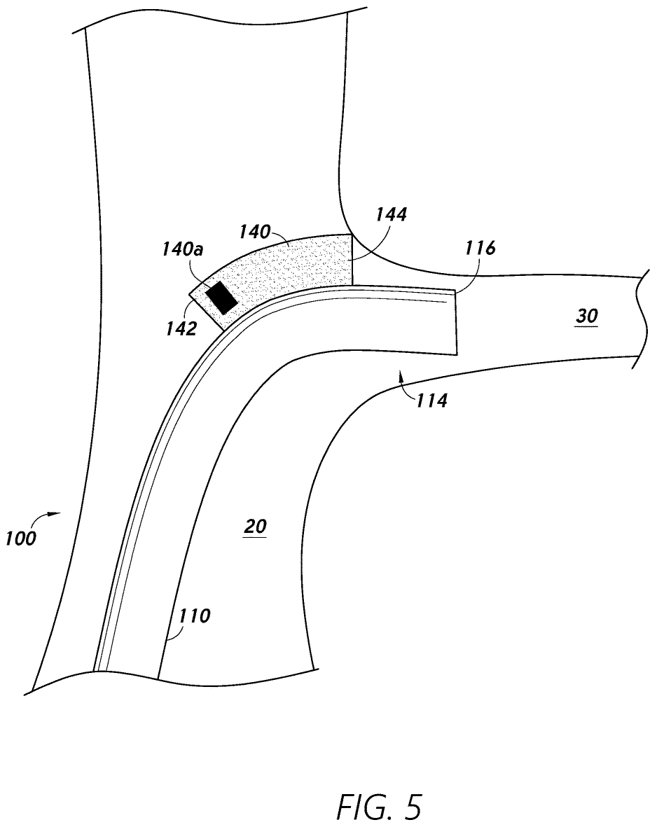

FIG. 5 is a schematic diagram showing the steerable sheath 100 positioned from a first body chamber or lumen 20 into a second body chamber or lumen 30. The first body chamber or lumen 20 and the second body chamber or lumen 30 can be any one of a number of fluid channels, blood vessels, and/or organ chambers. In some embodiments, the first body chamber or lumen 20 can be the right atrium and the second body chamber or lumen 30 can be the coronary sinus.

The distal portion 114 of the elongate tubular member 110 can have a bend while positioned from the first body chamber or lumen 20 into the second body chamber or lumen 30. The anchor 140 can be positioned at the opening of the second body chamber or lumen 30. The distal end 116 of the elongate tubular member 110 can extend into the second body chamber or lumen 30 while the anchor 140 is positioned at the opening of the second body chamber or lumen 30. As described herein, the anchor 140 can comprise one or more radiopaque markers 140a to facilitate visualization of the positioning of the anchor 140, the distal portion 114 and/or the distal end 116 of the elongate tubular member 110.

In some embodiments, one or more portions of the anchor 140 can be configured to engage with the opening of the second body chamber or lumen 30. In some embodiments, one or more portions of the anchor 140 can be configured to engage with both the opening of the second body chamber or lumen 30 and a portion of the second body chamber or lumen 30 adjacent to the opening. For example, the anchor 140 can have a proximal portion 142 and a distal portion 144, where the distal portion 144 of the anchor 140 can be configured to engage with the opening of the second body chamber or lumen 30. In some embodiments, the distal portion 144 of the anchor 140 can be configured to engage with both the opening of the second body chamber or lumen 30 and an adjacent portion of the second body chamber or lumen 30. It will be understood that although FIG. 5 shows the anchor 140 as being positioned outside of the second body chamber or lumen 30, the anchor 140 can be positioned at least partially within the second body chamber or lumen 30.

Engaging the anchor 140 with the opening of the second body chamber or lumen 30, or with both the opening of the second body chamber or lumen 30 and an adjacent portion of the second body chamber or lumen 30, can facilitate docking of the elongate tubular member 110 against the opening of the second body chamber or lumen 30, or both the opening of the second body chamber or lumen 30 and an adjacent portion of the second body chamber or lumen 30. As such, over insertion of the elongate tubular member 110 into the second body chamber or lumen 30, and/or undesired movement of the elongate tubular member 110, can be prevented or reduced.

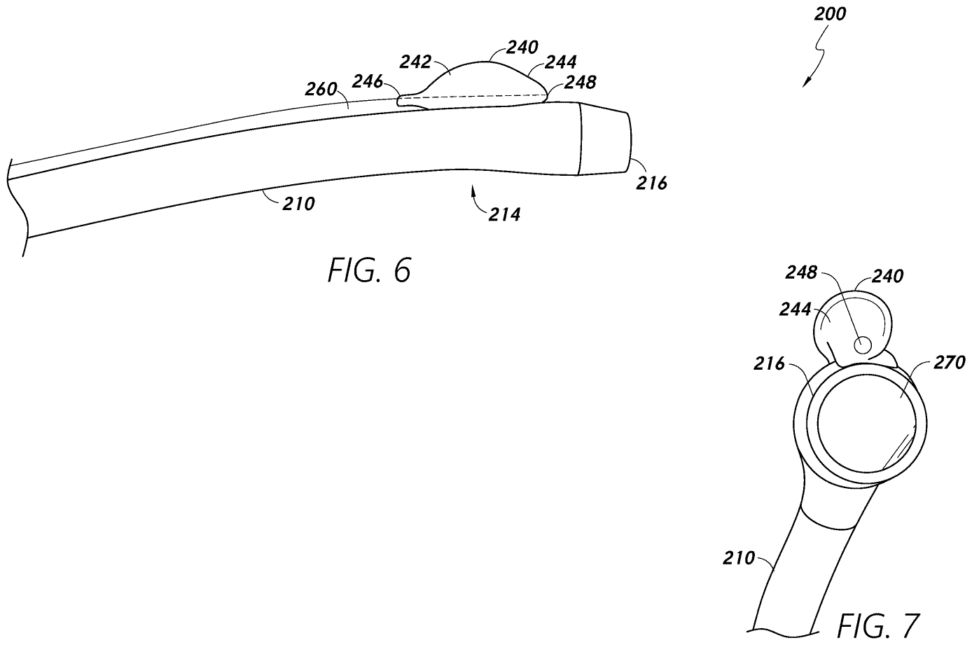

FIG. 6 is a side view of an example of a steerable sheath 200 comprising a partially circumferential balloon anchor 240. The steerable sheath 200 can comprise an elongate tubular member 210 and the partially circumferential balloon anchor 240 can be positioned around only a portion of a circumference of a distal portion 214 of the tubular member 210. As used herein, a partially circumferential balloon anchor can refer to any balloon anchor configured to be positioned on, disposed around and/or extending from, any partial portion of the circumference of the distal portion 214, and where the balloon anchor does not circumscribe the distal portion 214.

In some embodiments, the partially circumferential balloon anchor 240 can be inflatable. For example, the steerable sheath 200 can comprise an inflation and deflation lumen 260 extending along at least a portion of the elongate tubular member 210, the inflation and deflation lumen 260 being configured to allow inflation or deflation of the partially circumferential balloon anchor 240. The partially circumferential balloon anchor 240, when in an inflated state, can be configured to contact and engage with the coronary sinus ostium and a portion of the coronary sinus adjacent to the ostium, so as to facilitate reliable positioning of the elongate tubular member 210 within the coronary sinus.

The partially circumferential balloon anchor 240 can comprise a proximal end 246 and a distal end 248. The partially circumferential balloon anchor 240 can be positioned on the distal portion 214 of the elongate tubular member 210 such that both the proximal end 246 and the distal end 248 are on the distal portion 214. In some embodiments, the partially circumferential balloon anchor 240 can be positioned a distance away from a distal end 216 of the elongate tubular member 210. For example, the distal end 248 of the circumferential balloon anchor 240 can be spaced from the distal end 216 of the elongate tubular member 210 such that the circumferential balloon anchor 240 can be positioned at the coronary sinus ostium, while the distal end 216 of the elongate tubular member 210 can be positioned further within the coronary sinus, such as at or proximate to a target tissue location. In some embodiments, the distal end 248 of the partially circumferential balloon anchor 240 can be at the distal end 216 of the elongate tubular member 210.

In some embodiments, the partially circumferential balloon anchor 240 can be inflatable. In the inflated state, the partially circumferential balloon anchor 240 can comprise a shape configured to engage the coronary sinus ostium and a portion of the coronary sinus adjacent to the ostium. As described herein, a cross-sectional size of the coronary sinus decreases along a direction extending into the coronary sinus and away from the ostium. A distal portion 244 of the partially circumferential balloon anchor 240 can be configured to engage with the coronary sinus ostium and an adjacent portion of the coronary sinus. For example, the distal portion 244 can have a longitudinal profile comprising a taper toward the distal end 248 of the partially circumferential balloon anchor 240, the longitudinal profile extending along a direction parallel or substantially parallel to a longitudinal axis of the elongate tubular member 210. The taper of the longitudinal profile can be configured to facilitate desired engagement of the distal portion 244 with the coronary sinus ostium and a portion of the coronary sinus adjacent to the ostium. In some embodiments, the tapered distal portion 244 can comprise a curvature, such as a convex curvature. For example, the longitudinal profile of the distal portion 244 can comprise a curved taper, such as a curved convex taper, which tapers towards the distal end 248 of the partially circumferential balloon anchor 240. The longitudinal profile of the distal portion 244 can be selected to provide secure engagement of the partially circumferential balloon anchor 240 with the coronary sinus ostium and an adjacent portion of the coronary sinus.

In some embodiments, a proximal portion 242 of the partially circumferential balloon anchor 240 can comprise a shape configured to facilitate movement of the partially circumferential balloon anchor 240 through anatomical pathways, including during retraction of the partially circumferential balloon anchor 240. For example, the proximal portion 242 of the partially circumferential balloon anchor 240 can have a longitudinal profile comprising a taper toward the proximal end 246 of the partially circumferential balloon anchor 240, the longitudinal profile extending along a direction parallel or substantially parallel to a longitudinal axis of the elongate tubular member 210. The longitudinal profile of the proximal portion 242 can comprise a curved taper, such as a curved convex taper, which tapers towards the proximal end 246 of the partially circumferential balloon anchor 240. In some embodiments, the partially circumferential balloon anchor 240 can have a maximum cross-sectional size at or around a mid-portion of the anchor 240, and a longitudinal profile of both the proximal portion 242 and distal portion 244 comprising a taper, including a curved convex taper, from or around the mid-portion of the anchor 240 toward the proximal end 246 and the distal tend 248, respectively.

In some embodiments, the partially circumferential balloon anchor 240 can have a length of up to about 10 millimeters (mm), including about 1 mm to about 9 mm, about 2 mm to about 8 mm, or about 3 mm to about 7 mm. In some embodiments, the partially circumferential balloon anchor 240 has a length of about 10 mm. In some embodiments, a width of the partially circumferential balloon anchor 240 can be about 1 mm to about 10 mm. In some embodiments, the partially circumferential balloon anchor 240 can have a width which varies from about 1 mm to about 10 mm. For example, the distal portion 244 can taper from a width of about 10 mm to a width of about 1 mm at the distal end 248, including from about 9 mm down to about 1 mm, about 8 mm down to about 1 mm, about 9 mm down to about 2 mm, about 8 mm down to about 2 mm, about 7 mm down to about 3 mm, or about 6 mm down to about 4 mm. In some embodiments, the proximal portion 242 can have the same or similar taper in width.

In some embodiments, the partially circumferential balloon anchor 240 can be kept in a deflated state during at least a part of its insertion to and/or retraction from the coronary sinus ostium. In some embodiments, the partially circumferential balloon anchor 240 can assume an inflated state after the distal portion 214 of the elongate tubular member 210 is positioned at a desired location. For example, after the distal end 216 of the elongate tubular member 210 is positioned at or adjacent to the target tissue site and the partially circumferential balloon anchor 240 is at the coronary sinus ostium, the partially circumferential balloon anchor 240 can be inflated. The partially circumferential balloon anchor 240 can be inflated until desired engagement with the coronary sinus ostium and an adjacent portion of the coronary sinus is achieved. In some embodiments, the partially circumferential balloon anchor 240 can be inflated before the distal portion 214 of the elongate tubular member 210 is positioned at the desired location. For example, the partially circumferential balloon anchor 240 can assume an inflated state after being positioned within the right atrium but before being positioned to engage the coronary sinus ostium and an adjacent portion of the coronary sinus. In some embodiments, the partially circumferential balloon anchor 240 can be expanded both before and after being positioned at a target location. For example, the partially circumferential balloon anchor 240 can assume a partially inflated state while in the right atrium, and further inflation can be performed after being positioned to engage the coronary sinus ostium. In some embodiments, a degree of inflation of the partially circumferential balloon anchor 240 can be adjusted during a procedure in response to a size of a coronary sinus ostium and an adjacent portion of the coronary sinus. In some embodiments, the partially circumferential balloon anchor 240 can be deflated for retraction.

FIG. 7 is a view of the distal portion 214 of the elongate tubular member 210. The distal end 248 and the distal portion 244 of the partially circumferential balloon anchor 240 is shown. As shown in FIG. 7, the partially circumferential balloon anchor 240 can be positioned around only a portion of the circumference of the distal portion 214.

Having the partially circumferential balloon anchor 240 only around a portion of the circumference of the distal portion 214 can facilitate blood flow through the coronary sinus ostium and past the partially circumferential balloon anchor 240 while the partially circumferential balloon anchor 240 is positioned at and in engagement with the coronary sinus ostium and an adjacent portion of the coronary sinus. The partially circumferential balloon anchor 240 can provide sufficient contact with the coronary sinus ostium and an adjacent portion of the coronary sinus while allowing desired area around the steerable sheath 200 at the coronary sinus ostium such that sufficient blood flow can pass therethrough. In some embodiments, the partially circumferential balloon anchor 240 can be positioned around less than about 50% of the circumference of the distal portion 214 of the elongate tubular member 210, or less than about 40%, less than about 30%, less than about 25%, or less than about 20%.

As shown in FIG. 7, an inner lumen 270 of the elongate tubular member 210 extends to the distal end 216 of the elongate tubular member 210. The inner lumen 270 can be configured to receive various instrumentation for delivering one or more therapy devices to a target tissue location.

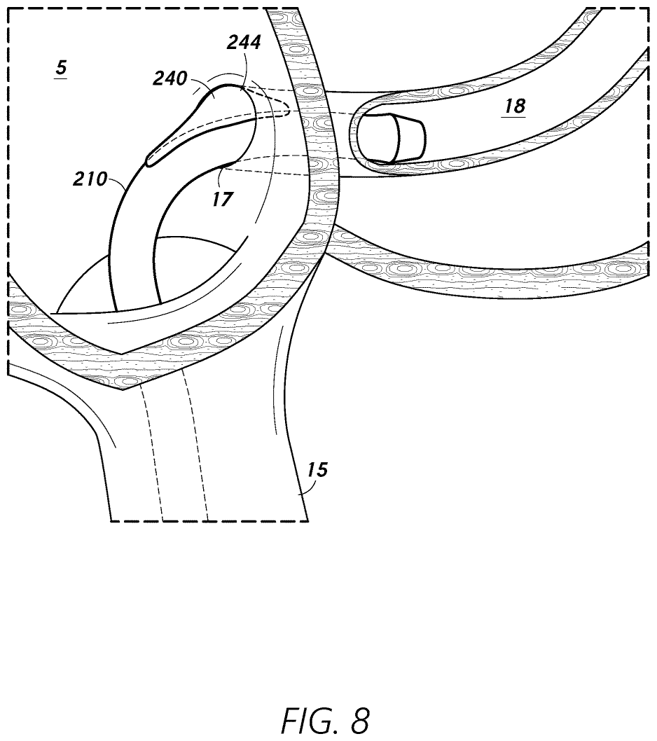

FIG. 8 shows the steerable sheath 200 in a position for accessing a target location within the coronary sinus 18. The elongate tubular member 210 can be advanced through the inferior vena cava 15, positioned into the right atrium 5, and inserted through the coronary sinus ostium 17 into the coronary sinus 18. The distal end (not shown) of the elongate tubular member 210 can be positioned within the coronary sinus 18. The partially circumferential balloon anchor 240 can be positioned against the coronary sinus ostium 17 such that the distal portion 244 engages the coronary sinus ostium 17 and a portion of the coronary sinus 18 adjacent to the ostium 17. The distal end of the partially circumferential balloon anchor 240 can be positioned within the coronary sinus 18. The tapering of the longitudinal profile of the distal portion 244 can facilitate stable and/or reliable engagement of the partially circumferential balloon anchor 240 with the coronary sinus ostium 17 and an adjacent portion of the coronary sinus 18.

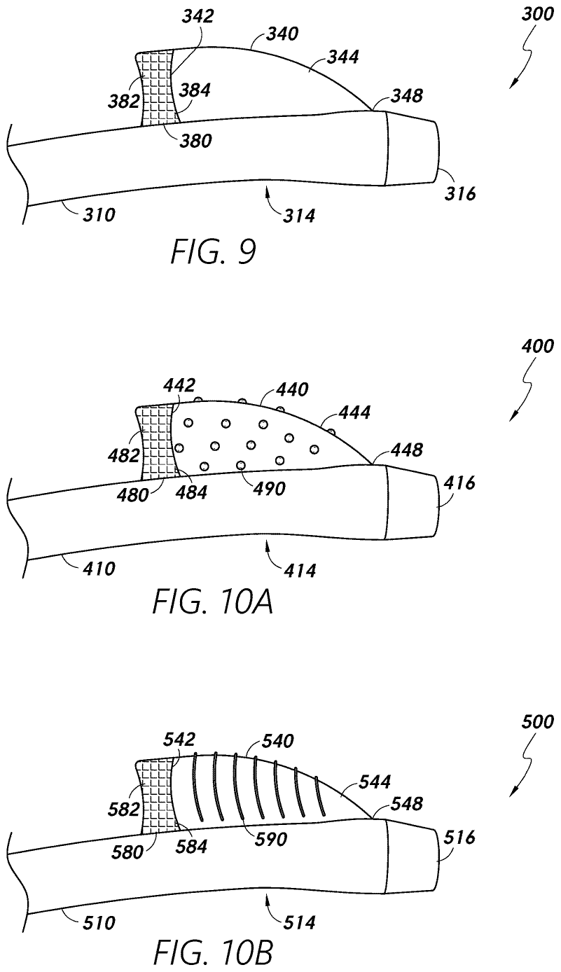

FIG. 9 is a schematic diagram of an example of a steerable sheath 300 comprising a partially circumferential balloon anchor 340 and a deformable panel 380 coupled thereto. The steerable sheath 300 can comprise an elongate tubular member 310. A distal portion 314 of the elongate tubular member 310 can comprise the partially circumferential balloon anchor 340 thereon.

In some embodiments, the partially circumferential balloon anchor 340 can comprise one or more features of the partially circumferential balloon anchor 240 described with reference to FIGS. 6 through 8. For example, the partially circumferential balloon anchor 340 can be inflatable. The partially circumferential balloon anchor 340, when in an inflated state, can be configured to contact and engage with the coronary sinus ostium and a portion of the coronary sinus adjacent to the ostium, so as to facilitate reliable positioning of the elongate tubular member 310 within the coronary sinus. For example, the distal portion 344 of the partially circumferential balloon anchor 340 can comprise a tapered longitudinal profile, the tapered longitudinal profile tapering toward the distal end 348 of the partially circumferential balloon anchor 340. The longitudinal profile can extend along a direction parallel or substantially parallel to a longitudinal axis of the elongate tubular member 310. In some embodiments, the tapered longitudinal profile can comprise a curve, including a convex curve. The distal end 348 of the circumferential balloon anchor 340 can be spaced from or at the distal end 316 of the elongate tubular member 310.

In some embodiments, the partially circumferential balloon anchor 340 can be positioned around less than about 50% of the circumference of the distal portion 314 of the elongate tubular member 310, or less than about 40%, or less than about 30%, or less than about 25%, or less than about 20%.

The proximal portion 342 of the partially circumferential balloon anchor 340 can extend laterally from the distal portion 314 of the elongate tubular member 310. The deformable panel 380 can be coupled to the proximal portion 342. The deformable panel 380 can extend laterally from the distal portion 314, including an example which extends perpendicularly or substantially perpendicularly from the distal portion 314. For example, the deformable panel 380 can comprise a proximal surface 382 and a distal surface 384. The distal surface 384 can be coupled to the proximal portion 342 of the balloon anchor 340. The deformable panel 380 can provide mechanical support, for example providing mechanical reinforcement, for the proximal portion 342. The deformable panel 380 can be configured to reduce deformation of an inflated partially circumferential balloon anchor 340, such as when the partially circumferential balloon anchor 340 engages the coronary sinus ostium and an adjacent portion of the coronary sinus. Reduced deformation of the inflated balloon anchor 340 can provide increased reliability in positioning of the elongate tubular member 310 within the coronary sinus.

FIGS. 10A and 10B are schematic diagrams of examples of two steerable sheaths each of which includes a partially circumferential balloon anchor comprising a textured surface thereon. FIG. 10A shows an example of a steerable sheath 400 including a partially circumferential balloon anchor 440 which comprises a plurality of bumps 490 on an exterior surface. FIG. 10B shows an example of a steerable sheath 500 including partially circumferential balloon anchor 540 which comprises a plurality of ridges 590 on an exterior surface.

The plurality of bumps 490 and the plurality of ridges 590 can be on a portion or all of the exterior surface of the partially circumferential balloon anchors 440, 540. In some embodiments, the plurality of bumps 490 and the plurality of ridges 590 can be only on the distal portion 444 or the distal portion 544, respectively. The plurality of bumps 490 and the plurality of ridges 590 can facilitate desired engagement of the balloon anchor 440 or the balloon anchor 540, respectively, with the coronary sinus ostium and an adjacent portion of the coronary sinus.

In some embodiments, a partially circumferential balloon anchor can comprise a textured exterior surface configured to engage with the coronary sinus ostium and an adjacent portion of the coronary sinus, the textured exterior surface comprising both a plurality of bumps and a plurality of ridges. It will also be understood that textured surfaces comprising one or more other patterns, in addition or in the alternative, can be applicable.

In some embodiments, the partially circumferential balloon anchors 440, 540 can comprise one or more features of the partially circumferential balloon anchor 340 described with reference to FIG. 9. For example, the partially circumferential balloon anchors 440, 540 can be inflatable. The partially circumferential balloon anchors 440, 540, when in an inflated state, can be configured to contact and engage with the coronary sinus ostium and a portion of the coronary sinus adjacent to the ostium, so as to facilitate reliable positioning of the elongate tubular members 410, 510 within the coronary sinus. The partially circumferential balloon anchors 440, 540 can be positioned on distal portions 414, 514 of the elongate tubular members 410, 510. The distal ends 448, 548 of the circumferential balloon anchors 440, 540 can be spaced from or at the distal ends 416, 516 of the elongate tubular members 410, 510. While inflated, the partially circumferential balloon anchors 440, 540 can comprise a shape to engage the coronary sinus ostium and a portion of the coronary sinus adjacent to the ostium. For example, the distal portions 444, 544 of the partially circumferential balloon anchors 440, 540 can comprise a tapered longitudinal profile, the tapered longitudinal profile tapering toward the distal ends 448, 548 of the partially circumferential balloon anchors 440, 540. The longitudinal profile can extend along a direction parallel or substantially parallel to a longitudinal axis of the elongate tubular members 410, 510. In some embodiments, the tapered longitudinal profile can comprise a curve, including a convex curve. The proximal portions 442, 542 of the partially circumferential balloon anchors 440, 540 can be coupled to deformable panels 480, 580. The deformable panels 480, 580 can comprise respective proximal surfaces 482, 582 and distal surfaces 484, 584. The distal surfaces 484, 584 can be coupled to the proximal portions 442, 542 of the partially circumferential balloon anchors 440, 540.

In some embodiments, the partially circumferential balloon anchors 440, 540 can be positioned around less than about 50% of the circumference of the distal portions 414, 514 of the elongate tubular members 410, 510, or less than about 40%, or less than about 30%, or less than about 25%, or less than about 20%.

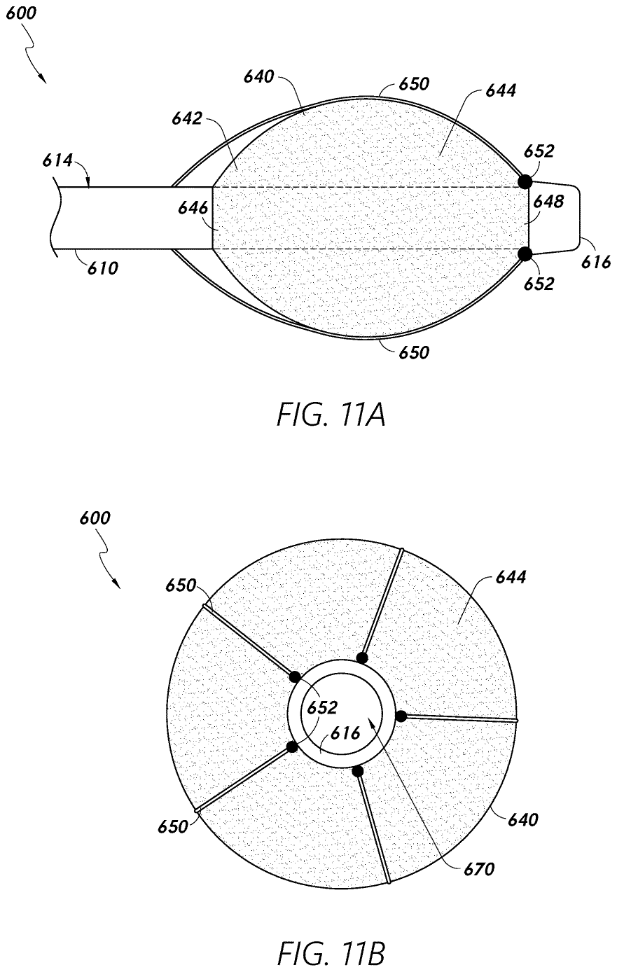

FIGS. 11A and 11B are schematic diagrams showing an example of a steerable sheath 600 comprising a circumferential balloon anchor 640 circumferentially disposed around an elongate tubular member 610 of the steerable sheath 600 and a plurality of cords 650 circumferentially distributed around the circumferential balloon anchor 640.

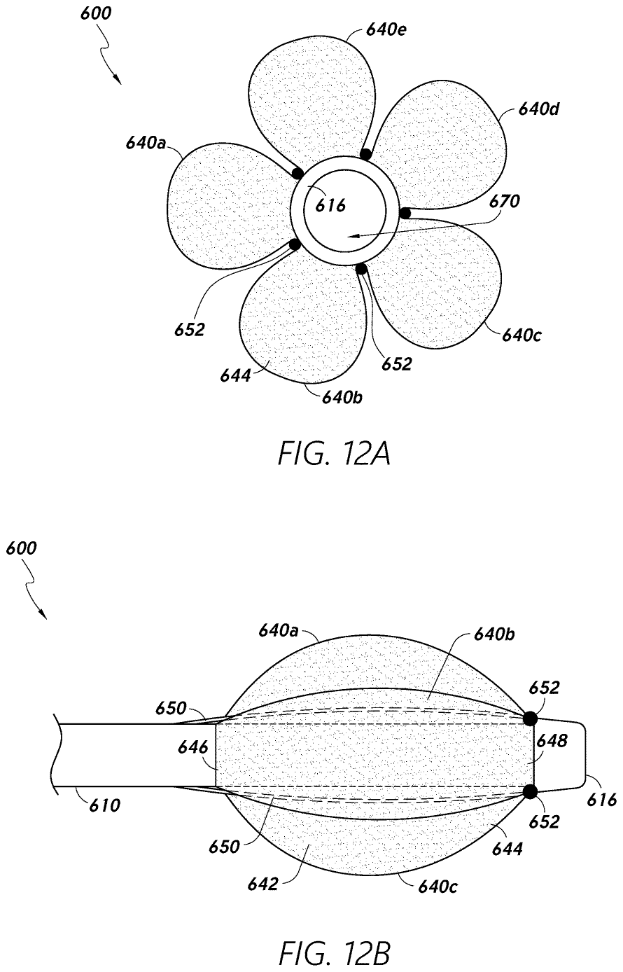

FIGS. 11A and 11B show an inflated circumferential balloon anchor 640. FIG. 11A is a side view of the steerable sheath 600 and FIG. 11B is a view of the steerable sheath 600 from a distal end 616 of the elongate tubular member 610. FIGS. 12A and 12B are schematic diagrams showing the plurality of cords 650 engaged with the inflated circumferential balloon anchor 640 to form the corresponding indentations on the inflated circumferential balloon anchor 640. FIG. 12A is a view of the steerable sheath 600 from the distal end 616 of the elongate tubular member 610. FIG. 12B is a side view of the steerable sheath 600. The elongate tubular member 610 can comprise an inner lumen 670 configured to receive instrumentation for delivering one or more therapy devices to a target location, such as a delivery catheter for carrying an atrial shunt device to a target location on the left atrial wall. The inflated circumferential balloon anchor 640 can be configured to engage with the coronary sinus ostium, or the coronary sinus ostium and an adjacent portion of the coronary sinus, while blood flow is permitted through the ostium. Blood flow through the ostium may be along one or more paths defined at least in part by one or more of the plurality of indentations. As shown in the figures, the circumferential balloon anchor 640 can be a distance from the distal end 616 of the elongate tubular member 610. For example, a distal end 648 of the circumferential balloon anchor 640 can be a distance proximal of the distal end 616 of the elongate tubular member 610. In some embodiments, the circumferential balloon anchor 640 can be at the distal end 616 of the elongate tubular member 610.

Referring to FIG. 11A, the circumferential balloon anchor 640 can be circumferentially positioned around a distal portion 614 of the elongate tubular member 610. Distal ends 652 of each of the plurality of cords 650 can be coupled to respective locations on the distal portion 644 of the circumferential balloon anchor 640. In some embodiments, distal ends 652 of each of the plurality of cords 650 can be coupled to respective locations on the distal portion 614 of the elongate tubular member 610. Each of the plurality of cords 650 can comprise a portion which extends over at least a portion of the circumferential balloon anchor 640, extending along a longitudinal axis of the elongate tubular member 610.

Referring to FIG. 11B, respective distal ends 652 of the plurality of cords 650 can be coupled to corresponding locations on the distal portion 644 of the circumferential balloon anchor 640 and be distributed around the circumferential balloon anchor 640. The circumferential balloon anchor 640 can have a longitudinal axis symmetry, for example being symmetrical around an axis parallel or substantially parallel to the longitudinal axis of the elongate tubular member 610. In some embodiments, the plurality of cords 650 can be evenly or substantially evenly distributed around the circumferential balloon anchor 640, for example dividing the circumferential balloon anchor 640 into even or substantially even portions. FIG. 11B shows five cords 650 positioned around the balloon circumferential anchor 640. For example, the five cords 650 can be evenly or substantially evenly distributed around the circumferential balloon anchor 640 to divide the circumferential balloon anchor into five equal or substantially equal portions. In some embodiments, the plurality of cords 650 may not be evenly distributed around the circumferential balloon anchor 640.

The plurality of cords 650 can engage an inflated circumferential balloon anchor 640 to form corresponding indentations on the inflated circumferential balloon anchor 640. Referring to FIG. 12A, the plurality of indentations can divide the circumferential balloon anchor 640 into a plurality of portions. For example, the five cords 650 can divide the inflated circumferential balloon anchor 640 into five portions 640a, 640b, 640c, 640d, 640e. In some embodiments, the five cords 650 can be evenly or substantially evenly distributed around the balloon anchor 640 such that the five portions 640a, 640b, 640c, 640d, 640e are uniform or substantially uniform in size and/or shape.

The circumferential balloon anchor 640 can be inflated, and the plurality of cords 650 can be tensioned such that the plurality of cords 650 can compress the inflated circumferential balloon anchor 640 to form the corresponding plurality of indentations. The circumferential balloon anchor 640 can be inflated before and/or after being positioned at the coronary sinus ostium. After the circumferential balloon anchor 640 is in the inflated state, the plurality of cords 650 can be moved proximally relative to the elongate tubular member 610, for example proximally along a longitudinal axis of the elongate tubular member 610, to tension the plurality of cords 650. In some embodiments, the plurality of cords 650 can be in a tensioned state prior to inflation of the circumferential balloon anchor 640 such that inflating the circumferential balloon anchor 640 results in formation of the corresponding plurality of indentations. Pre-tensioning the plurality of cords 650 can reduce needed manipulation of the steerable sheath 600 during a procedure, providing a simplified procedure.

In some embodiments, the circumferential balloon anchor 640 can be configured to engage the ostium of the coronary sinus. In some embodiments, the circumferential balloon anchor 640 can be configured to engage the ostium and an adjacent portion of the coronary sinus. Referring to FIG. 12B, in some embodiments, the distal portion 644 of the circumferential balloon anchor 640 can have a longitudinal profile comprising a taper toward the distal end 648 of the circumferential balloon anchor 640, the longitudinal profile extending along a direction parallel or substantially parallel to the longitudinal axis of the elongate tubular member 610. The taper of the longitudinal profile can be configured to facilitate desired engagement of the distal portion 644 with the coronary sinus ostium and/or the portion of the coronary sinus adjacent to the ostium. In some embodiments, the tapered distal portion 644 can comprise a curvature, such as a convex curvature. In some embodiments, a proximal portion 642 of the circumferential balloon anchor 640 can comprise a taper to facilitate movement of the circumferential balloon anchor 640, including retraction of the circumferential balloon anchor 640. The longitudinal profile of the proximal portion 642 can comprise a curved taper, such as a curved convex taper, which tapers towards the proximal end 646 of the circumferential balloon anchor 640. In some embodiments, the circumferential balloon anchor 640 can have a maximum cross-sectional size at or around a mid-portion of the anchor 640, and a longitudinal profile of both the proximal portion 642 and distal portion 644 comprising a taper, including a curved convex taper, from or around the mid-portion of the anchor 640 toward the proximal end 646 and the distal tend 648, respectively.

In some embodiments, a circumferential balloon anchor can comprise a textured exterior surface configured to engage with the coronary sinus ostium, or the coronary sinus ostium and an adjacent portion of the coronary sinus. The textured exterior surface can comprise one or both of a plurality of bumps and a plurality of ridges. It will be understood that textured surfaces comprising one or more other patterns, in addition or in the alternative, can be applicable.

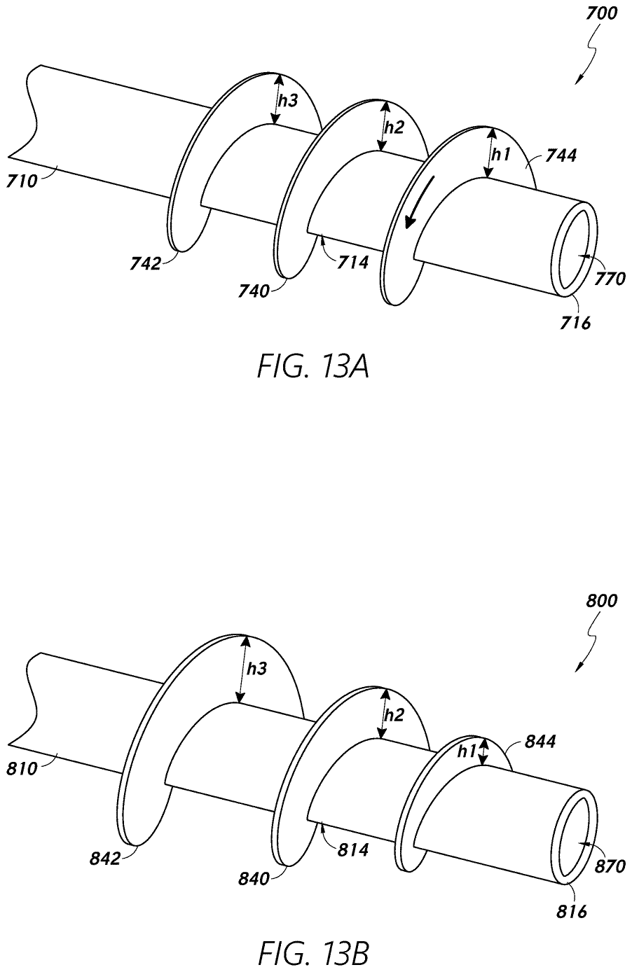

FIGS. 13A and 13B show examples of steerable sheaths comprising helical rib anchors. FIG. 13A is a perspective view of an example of a steerable sheath 700 comprising a helical rib anchor 740 disposed around a distal portion 714 of an elongate tubular member 710. FIG. 13B is a perspective view of another example of a steerable sheath 800 comprising a helical rib anchor 840 disposed around a distal portion 814 of an elongate tubular member 810. The helical rib anchors 740, 840 can be configured to engage with the coronary sinus ostium and a portion of the coronary sinus adjacent to the ostium. In some embodiments, the helical rib anchors 740, 840 can be deformable to facilitate insertion and/or retraction, and/or secure engagement with the coronary sinus ostium and an adjacent portion of the coronary sinus. The helical rib anchors 740, 840 can be a distance from or at respective distal ends 716, 816 of the elongate tubular members 710, 810 (e.g., distal ends 744, 844 of the helical rib anchors 740, 840 can be a distance from or at respective distal ends 716, 816 of the elongate tubular members 710, 810). The elongate tubular members 710, 810 can comprise respective inner lumens 770, 870 configured to receive instrumentation for delivering one or more therapy devices to a target location, such as a delivery catheter for carrying an atrial shunt device to a target location on the left atrial wall.

At least a portion of the helical anchors 740, 840 can be configured to be positioned within the coronary sinus. In some embodiments, one or both of the helical rib anchors 740, 840 can be configured to be entirely or substantially entirely positioned within the coronary sinus, both the distal ends 744, 844 and proximal ends 742, 842 being configured to be positioned within the coronary sinus. For example, the proximal ends 742, 842 can be configured to engage with the ostium. In some embodiments, one or both of the helical rib anchors 740, 840 can be configured to be only partially positioned within the coronary sinus. For example, the distal ends 744, 844 and not the proximal ends 742, 842 can be configured to be positioned within the coronary sinus. In some embodiments, an extent to which the helical rib anchors 740, 840 can be positioned into a coronary sinus can be based on individual anatomy.

The helical rib anchors 740, 840 can be configured to allow blood flow through the coronary sinus ostium while the helical rib anchors 740, 840 are in their respective target positions. Blood can flow along a path defined at least in part by the helical rib anchors 740, 840. For example, blood can flow along the helical ribs, such as shown by the arrows in FIGS. 13A and 13B. While the helical rib anchors 740, 840 are positioned at their respective target locations, blood can flow along a path defined at least in part by the helical ribs of the helical anchors 740, 840 through the coronary sinus ostium.

Referring to FIG. 13A, a height at which the helical rib anchor 740 extends from the elongate tubular member 710 can be uniform or substantially uniform along a dimension parallel or substantially parallel to a longitudinal axis of the elongate tubular member 710. For example, a first height, h1, a second height, h2, and a third height, h3, of the helical rib anchor 740 shown in FIG. 13A can be the same or substantially the same.

Referring to FIG. 13B, a height at which the helical rib anchor 840 extends from the elongate tubular member 810 can increase along a dimension parallel or substantially parallel to a longitudinal axis of the elongate tubular member 810, for example increasing with increased distance from the distal end 816 of the elongate tubular member 810. In FIG. 13B, a first height, h1, of the helical rib anchor 840 can be smaller than a second height, h2, which can be smaller than a third height, h3. In some embodiments, a decreasing height of the helical rib anchor 840 toward the distal end 816 of the elongate tubular member 810 can be configured to provide improved engagement with the coronary sinus ostium and an adjacent portion of the coronary sinus, such as by conforming to the anatomy of the coronary sinus.

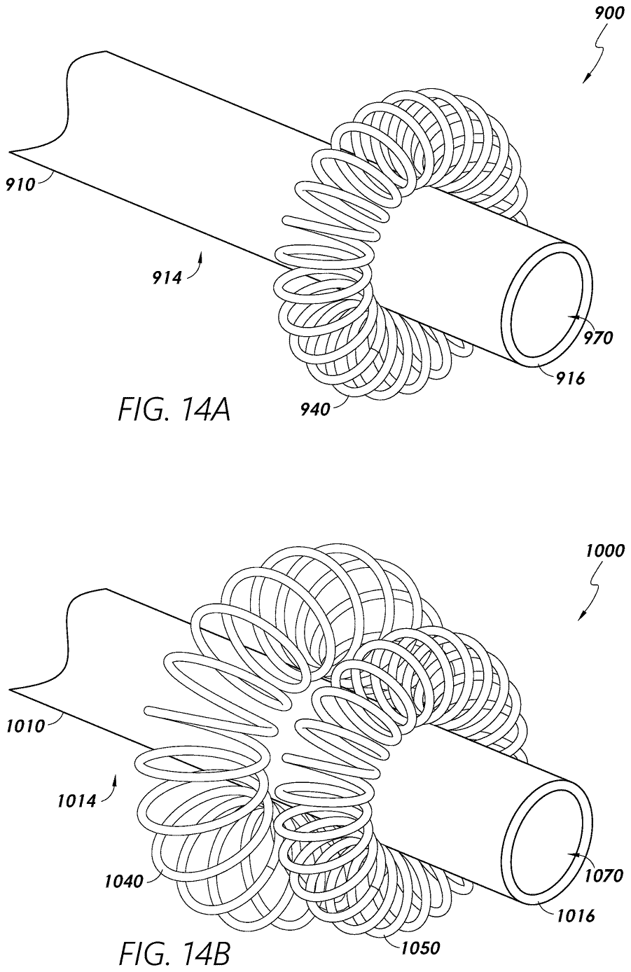

FIGS. 14A and 14B show examples of steerable sheaths comprising one or more coil anchors. FIG. 14A is a perspective view of an example of a steerable sheath 900 comprising a coil anchor 940 disposed around a distal portion 914 of an elongate tubular member 910. FIG. 14B is a perspective view of an example of a steerable sheath 1000 comprising a first coil anchor 1040 and a second coil anchor 1050 disposed around a distal portion 1014 of an elongate tubular member 1010. Each of the elongate tubular members 910, 1010 can comprise an inner lumen 970, 1070 configured to receive instrumentation for delivering one or more therapy devices to a target location. For example, the inner lumens 970, 1070 can be configured to receive a delivery catheter for carrying an atrial shunt device to a target location on the left atrial wall.

The coil anchor 940 of the steerable sheath 900 can be configured to engage with the coronary sinus ostium. The coil anchor 940 can be positioned at the ostium while a distal end 916 of the elongate tubular member 910 can be at or proximate to a target tissue site on the left atrial wall accessible from within the coronary sinus. For example, the coil anchor 940 can be at a distance from the distal end 916 of the elongate tubular member 910 such that the distal end 916 can be positioned into the coronary sinus while at least a portion of the coil anchor 940 is in the ostium. In some embodiments, the coil anchor 940 can be positioned to engage the ostium such that a portion of the coil anchor 940 is in the right atrium and a portion of the coil anchor 940 is in the coronary sinus. The coil anchor 940 can be configured to permit blood to flow through the ostium while the coil anchor 940 is in desired engagement with the ostium. For example, blood can flow between the coils of the coil anchor 940.

Referring to FIG. 14B, the first coil anchor 1040 of the steerable sheath 1000 can be positioned adjacent to and proximal of second coil anchor 1050 on the distal portion 1014 of the elongate tubular member 1010. The second coil anchor 1050 can include coils comprising a diameter smaller than that of the first coil anchor 1040. The first coil anchor 1040 can be configured to engage the coronary sinus ostium and the second coil anchor 1050 can be configured to engage a portion of the coronary sinus adjacent to the ostium. For example, the first coil anchor 1040 and the second coil anchor 1050 can be at a distance from the distal end 1016 of the elongate tubular member 1010 such that the distal end 1016 can be positioned within the coronary sinus while the first coil anchor 1040 and the second coil anchor 1050 engage the coronary sinus ostium and an adjacent portion of the coronary sinus, respectively. In some embodiments, the second coil anchor 1050 can be at the distal end 1016 of the elongate tubular member 1010.

The coil anchors 1040, 1050 can be configured to permit blood to flow through the ostium while the coil anchors 1040, 1050 are in desired engagement with the coronary sinus ostium and an adjacent portion of the coronary sinus. For example, blood can flow through the ostium between coils of the coil anchors 1040, 1050.

In some embodiments, one or more of the coil anchors 940, 1040, 1050 can be expandable. One or more of the coil anchors 940, 1040, 1050 can be kept in a compact state during at least a part of the insertion of the elongate tubular members 910, 1010 to the target tissue site. In some embodiments, one or more of the coil anchors 940, 1040, 1050 can comprise shape memory material. The one or more coil anchors 940, 1040, 1050 can be maintained in the compact state until a predetermined location is reached. The one or more coil anchors 940, 1040, 1050 can then be allowed to assume an expanded state, such as when the one or more coil anchors 940, 1040, 1050 comprising the shape memory material can assume their respective desired coil configurations. In some embodiments, a diameter of the coils of one or more of the coil anchors 940, 1040, 1050 can be adjusted based on patient anatomy. For example, a degree of expansion of the one or more coil anchors 940, 1040, 1050 can be adjusted during an implantation procedure in response to a size of a coronary sinus ostium, and/or an adjacent portion of the coronary sinus.

In some embodiments, the one or more of the coil anchors 940, 1040, 1050 can assume an expanded state after the coil anchors 940, 1040, 1050 are positioned at respective target locations. For example, after the distal ends 916, 1016 of the elongate tubular members 910, 1010 are positioned at or adjacent to the target tissue site, one or more of the coil anchors 940, 1040, 1050 can assume the expanded state. In some embodiments, the one or more of the coil anchors 940, 1040, 1050 can be expanded until desired engagement with the coronary sinus ostium and/or an adjacent portion of the coronary sinus is achieved. In some embodiments, one or more of the coil anchors 940, 1040, 1050 can assume an expanded state before the coil anchors 940, 1040, 1050 are positioned at respective target locations. For example, the one or more of the coil anchors 940, 1040, 1050 can be expanded after the one or more of the coil anchors 940, 1040, 1050 are extended from the inferior vena cava or the superior vena cava into the right atrium but before the one or more of the coil anchors 940, 1040, 1050 are positioned at their respective desired locations. In some embodiments, the one or more of the coil anchors 940, 1040, 1050 can be expanded both before and after the coil anchors 940, 1040, 1050 are positioned at respective target locations. For example, the one or more of the coil anchors 940, 1040, 1050 can assume a partially expanded state before being positioned at the respective desired locations, such as while the one or more of the coil anchors 940, 1040, 1050 are in the right atrium. The coil anchors 940, 1040, 1050 can then assume a fully expanded state after the one or more of the coil anchors 940, 1040, 1050 are positioned at their respective desired locations.

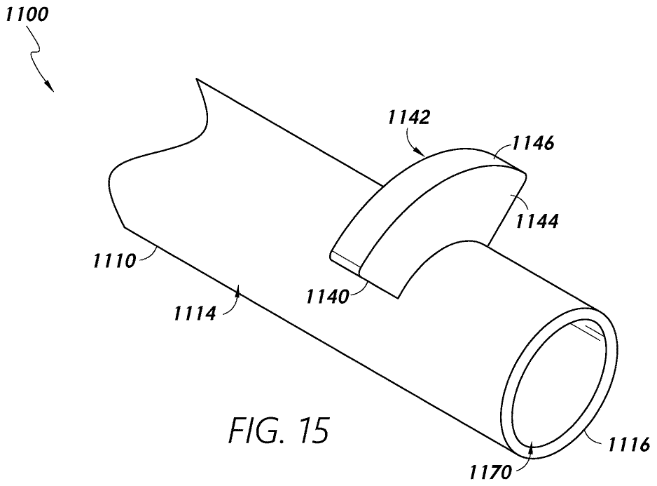

FIG. 15 is a perspective view of an example of a steerable sheath 1100 comprising a partially circumferential ring anchor 1140 positioned on an elongate tubular member 1110 of the steerable sheath 1100. The elongate tubular member 1110 can comprise an inner lumen 1170 configured to receive instrumentation for delivering one or more therapy devices to a target location, such as a delivery catheter for carrying an atrial shunt device to a target location on the left atrial wall. The partially circumferential ring anchor 1140 can be positioned on a distal portion 1114 of the elongate tubular member 1110. The partially circumferential ring anchor 1140 can be configured to be positioned to engage with the coronary sinus ostium. The partially circumferential ring anchor 1140 can be a distance from or at the distal end 1116 of the elongate tubular member 1110 (e.g., a distal portion 1144 of the partially circumferential ring anchor 1140 can be a distance from or at the distal end 1116 of the elongate tubular member 1110).

As shown in FIG. 15, the partially circumferential ring anchor 1140 can be positioned around only a portion of the circumference of the distal portion 1114. As used herein, a “partially circumferential ring anchor” can refer to any ring anchor configured to be positioned on, disposed around and/or extending from, any partial portion of the circumference of the distal portion 1114, and where the ring anchor does not circumscribe the distal portion 1114.

The partially circumferential ring anchor 1140 can facilitate blood flow through the coronary sinus ostium and past the partially circumferential ring anchor 1140 while the partially circumferential ring anchor 1140 is positioned at and in engagement with the coronary sinus ostium, or the coronary sinus ostium and a portion of the coronary sinus adjacent to the ostium. The partially circumferential ring anchor 1140 can provide desired contact with the coronary sinus ostium, or the coronary sinus ostium and a portion of the coronary sinus adjacent to the ostium, to provide stable positioning of the elongate tubular member 1110, while allowing sufficient area around the steerable sheath 1100 at the coronary sinus ostium such that sufficient blood flow can pass therethrough. In some embodiments, the partially circumferential ring anchor 1140 can be positioned around less than about 50% of the circumference of the distal portion 1114 of the elongate tubular member 1110, or less than about 40%, less than about 30%, less than about 25%, or less than about 20%.

The partially circumferential ring anchor 1140 can have a proximal portion 1142, a distal portion 1144 and a lateral portion 1146 extending between the proximal portion 1142 and the distal portion 1144. In some embodiments, the distal portion 1144 and/or the lateral portion 1146 can be configured to engage with the coronary sinus ostium, or both the coronary sinus ostium and a portion of the coronary sinus adjacent to the ostium. In some embodiments, at least a portion of the lateral portion 1146 can be configured to engage the ostium, or the ostium and a portion of the coronary sinus adjacent to the ostium. For example, the partially circumferential ring anchor 1140 can be sized such that when at least a portion of the anchor 1140 is positioned within the coronary sinus ostium, the lateral portion 1146 can be configured to engage the coronary sinus ostium, or the coronary sinus ostium and an adjacent portion, to provide stable positioning of the elongate tubular member 1110.

In some embodiments, the distal portion 1144 can be configured to engage the ostium. In some embodiments, the partially circumferential ring anchor 1140 can be positioned against the ostium such that the distal portion 1144 is configured to engage the ostium. For example, the partially circumferential ring anchor 1140 can be positioned against the coronary sinus ostium such that the proximal portion 1142 and the lateral portion 1146 of the partially circumferential ring anchor 1140 are outside or substantially outside of the coronary sinus, and the distal portion 1144 can engage the coronary sinus ostium.

In some embodiments, a partially circumferential ring anchor can comprise a planar or substantially planar distal portion and/or proximal portion. In some embodiments, the distal portion and/or the proximal portion can be non-planar. In some embodiments, a distal portion of a partially circumferential ring anchor can comprise a shape and/or dimension to engage the coronary sinus ostium, or the coronary sinus ostium and an adjacent portion of the coronary sinus ostium. For example, the distal portion can have a longitudinal profile comprising a taper toward a distal end of the partially circumferential ring anchor. The longitudinal profile can extend along a direction parallel or substantially parallel to a longitudinal axis of the elongate tubular member. The tapered longitudinal profile may comprise a curved taper, such as a convex taper, configured to engage the coronary sinus ostium and an adjacent portion of the coronary sinus. In some embodiments, the lateral portion can be configured to engage the coronary sinus ostium and the distal portion can be configured to engage a portion of the coronary sinus adjacent to the ostium.

In some embodiments, a steerable sheath can comprise a suction anchor. The steerable sheath can comprise an elongate tubular member, and the suction anchor can be positioned on a distal portion of the elongate tubular member. The suction anchor can be configured to engage with the coronary sinus ostium, or the coronary sinus ostium and an adjacent portion, and permit blood to flow through the ostium, while the suction anchor is positioned at the ostium.

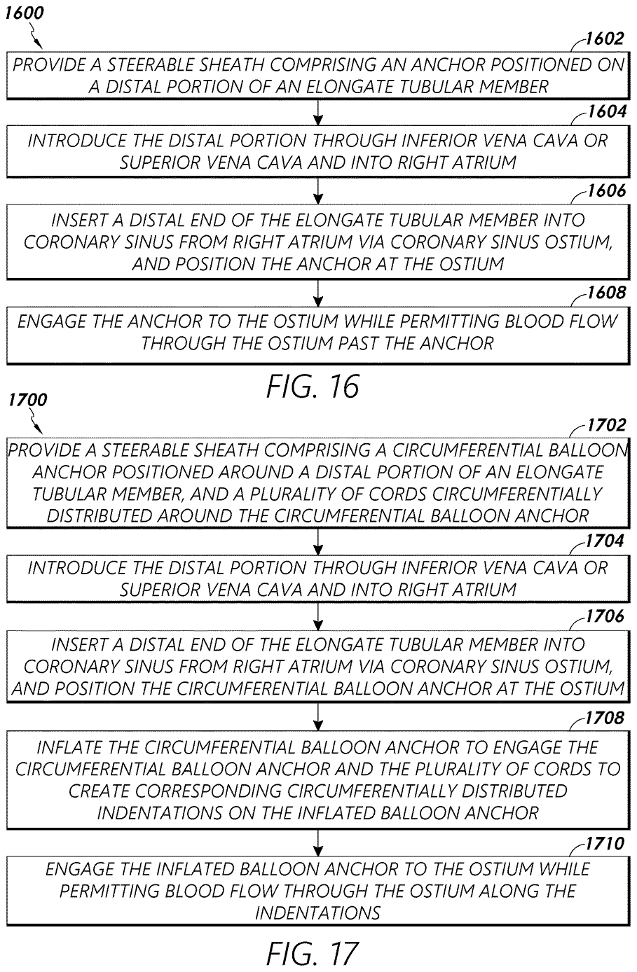

FIG. 16 is a flow diagram of an example of a procedure 1600 for deploying a steerable sheath into the coronary sinus. The steerable sheath can comprise an elongate tubular member and an anchor positioned on a distal portion of the elongate tubular member. The steerable sheath can have one or more configurations as described herein. For example, the anchor can comprise any one of the partially circumferential balloon anchor, circumferential balloon anchor, helical rib anchor, coil anchor, partially circumferential ring anchor, and suction anchor, as described herein.

In block 1602, the procedure 1600 involves providing the steerable sheath. In block 1604, the procedure 1600 involves introducing the distal portion of the elongate tubular member through the inferior vena cava or superior vena cava, and into the right atrium. In some embodiments, the elongate tubular member can be introduced through a femoral vein and into the inferior vena cava. In some embodiments, the elongate tubular member can be introduced through a jugular vein or a subclavian vein and into the superior vena cava. In block 1606, the procedure 1600 involves inserting the distal end of the elongate tubular member into the coronary sinus from the right atrium via the coronary sinus ostium, and the anchor can be positioned at the coronary sinus ostium. The anchor can be on the distal portion of the elongate tubular member and at a distance from a distal end of the elongate tubular member. The anchor can be spaced from the distal end of the elongate tubular member such that while the distal end is placed at or adjacent to a target tissue site, the anchor can be positioned to engage with the coronary sinus ostium. In some embodiments, the anchor can be at the distal end of the elongate tubular member.

In block 1608, the procedure 1600 involves engaging the anchor with the coronary sinus ostium, while blood flow is permitted through the ostium past the anchor. In some embodiments, the anchor can be engaged with both the coronary sinus ostium and a portion of the coronary sinus adjacent to the ostium.

FIG. 17 is a flow diagram of an example of a procedure 1700 for deploying a steerable sheath comprising a circumferential balloon anchor into the coronary sinus. The steerable sheath can comprise an elongate tubular member and a circumferential balloon anchor positioned on a distal portion of the elongate tubular member. A plurality of cords can be circumferentially distributed around the circumferential balloon anchor.

Respective distal ends of the plurality of cords can be coupled to corresponding locations on a distal portion of the circumferential balloon anchor. In some embodiments, respective distal ends of the plurality of cords can be coupled to corresponding locations on the distal portion of the elongate tubular member. In block 1702, the procedure 1700 involves providing the steerable sheath. In block 1704, the procedure 1700 involves introducing the distal portion of the elongate tubular member through the inferior vena cava or superior vena cava and into the right atrium. The circumferential balloon anchor can be on the distal portion of the elongate tubular member at a distance away from a distal end of the elongate tubular member. In some embodiments, the circumferential balloon anchor can be at the distal end of the elongate tubular member. In block 1706, the procedure 1700 involves inserting the distal end of the elongate tubular member into the coronary sinus from the right atrium via the coronary sinus ostium, and the circumferential balloon anchor can be positioned at the coronary sinus ostium.

In block 1708, the procedure 1700 involves inflating the circumferential balloon anchor to engage the circumferential balloon anchor with the plurality of cords to create a plurality of corresponding circumferentially distributed indentations around the inflated circumferential balloon anchor. The plurality of cords can be tensioned such that the plurality of cords can engage the inflated circumferential balloon anchor to provide the corresponding circumferentially distributed indentations. For example, the plurality of cords can be moved proximally in a direction along a longitudinal axis of the elongate tubular member to tension the plurality of cords such that the plurality of cords compress the inflated circumferential balloon anchor. The plurality of indentations can be formed by compression of the inflated circumferential balloon anchor using the plurality of cords.

The circumferential balloon anchor can be inflated both before and/or after the distal portion of the elongate tubular member is positioned at a desired location. The circumferential balloon anchor can be kept in a deflated state during at least a part its insertion to the coronary sinus ostium. In some embodiments, the circumferential balloon anchor can be inflated after being positioned at the coronary sinus ostium. The circumferential balloon anchor can be inflated until desired engagement with the coronary sinus ostium, or the coronary sinus ostium and an adjacent portion of the coronary sinus, is achieved. In some embodiments, the circumferential balloon anchor can be inflated after the circumferential balloon anchor is inserted into the right atrium but before the circumferential balloon anchor is positioned at the coronary sinus ostium. In some embodiments, a degree of inflation of the circumferential balloon anchor can be adjusted in response to individual anatomy.

In block 1710, the procedure 1700 involves engaging the inflated circumferential balloon anchor with the coronary sinus ostium, while blood flow is permitted through the ostium along the plurality of indentations. In some embodiments, the circumferential balloon anchor can be engaged with both the coronary sinus ostium and a portion of the coronary sinus adjacent to the ostium. In some embodiments, the circumferential balloon anchor can be deflated for retraction after the medical implant device and/or therapeutic procedure has been delivered.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

1. A steerable sheath comprising:
an elongate tubular member comprising an inner lumen configured to receive a plurality of instruments; and
a partially circumferential balloon anchor disposed partially circumferentially on a distal portion of the elongate tubular member and the partially circumferential balloon anchor including a proximal balloon portion and a distal balloon portion;
wherein, when the partially circumferential balloon anchor is in an inflated state the distal balloon portion has a tapered longitudinal profile tapering toward a distal end of the partially circumferential balloon anchor, and the partially circumferential balloon anchor is configured to, while positioned to engage with a coronary sinus, permit blood flow through an ostium of the coronary sinus.

2. The steerable sheath of claim 1, wherein the partially circumferential balloon anchor, in the inflated state, is configured to be positioned to engage with both the ostium and a portion of the coronary sinus adjacent to the ostium.

3. The steerable sheath of claim 1, wherein the tapered longitudinal profile comprises a concave curvature.

4. The steerable sheath of claim 1, wherein the partially circumferential balloon anchor is positioned around less than 50% of a circumference of the distal portion of the elongate tubular member.

5. The steerable sheath of claim 1, wherein the partially circumferential balloon anchor comprises a textured exterior surface configured to engage with the ostium and a portion of the coronary sinus adjacent to the ostium.

6. The steerable sheath of claim 5, wherein the textured exterior surface comprises a plurality of bumps.

7. The steerable sheath of claim 5, wherein the textured exterior surface comprises a plurality of ridges.

8. The steerable sheath of claim 1, wherein the proximal balloon portion extends laterally from the distal portion of the elongate tubular member, a deformable panel being coupled to the proximal balloon portion.

9. The steerable sheath of claim 1, wherein the inner lumen is configured to receive a therapy device delivery catheter carrying a therapy device for deployment to a heart location.

10. The steerable sheath of claim 9, wherein the therapy device comprises a shunt device for deployment onto a left atrial wall.

11. A steerable sheath comprising:
an elongate tubular member comprising an inner lumen configured to receive a plurality of instruments;
a circumferential balloon anchor disposed circumferentially around a distal portion of the elongate tubular member, and
a plurality of cords circumferentially distributed around the circumferential balloon anchor longitudinally; the plurality of cords configured to move proximally relative to the elongate tubular member along a longitudinal axis of the elongate tubular member to tension the plurality of cords to engage the circumferential balloon anchor in an inflated state to create corresponding circumferentially distributed longitudinal indentations on the circumferential balloon anchor,
wherein, the circumferential balloon anchor is configured to, while positioned to engage with a coronary sinus, permit blood flow through an ostium of the coronary sinus along a path defined at least in part by the circumferentially distributed longitudinal indentations.

12. The steerable sheath of claim 11, wherein the circumferential balloon anchor, in the inflated state, is configured to be positioned to engage with the ostium.

13. The steerable sheath of claim 11, wherein the circumferential balloon anchor comprises a proximal balloon portion and a distal balloon portion, and respective distal ends of the plurality of cords are coupled to corresponding locations on the distal balloon portion.

14. The steerable sheath of claim 13, wherein the distal balloon portion comprises a tapered longitudinal profile tapering toward a distal end of the circumferential balloon anchor, the distal balloon portion being configured to engage with the ostium and a portion of the coronary sinus adjacent to the ostium.

15. The steerable sheath of claim 11, wherein the circumferential balloon anchor comprises a textured exterior surface configured to engage with the ostium and a portion of the coronary sinus adjacent to the ostium.

16. The steerable sheath of claim 15, wherein the textured exterior surface comprises a plurality of bumps.

17. The steerable sheath of claim 15, wherein the textured exterior surface comprises a plurality of ridges.

18. The steerable sheath of claim 11, wherein the inner lumen is configured to receive a therapy device delivery catheter carrying a therapy device for deployment to a heart location.

19. The steerable sheath of claim 18, wherein the therapy device comprises a shunt device for deployment onto a left atrial wall.

20. A steerable sheath comprising:
an elongate tubular member comprising an inner lumen configured to receive a plurality of instruments;
a circumferential balloon anchor disposed circumferentially around a distal portion of the elongate tubular member, and
a plurality of cords circumferentially distributed around the circumferential balloon anchor longitudinally; the plurality of cords being configured to move proximally relative to the elongate tubular member along a longitudinal axis of the elongate tubular member to tension the plurality of cords against the circumferential balloon anchor when inflated to create corresponding circumferentially distributed longitudinal indentations on an outer surface of the circumferential balloon anchor,
wherein, the circumferentially distributed longitudinal indentations are configured to define a plurality of paths for fluid flow around the outer surface of the circumferential balloon anchor.
